(19) United States
(12) Patent Application Publication
(10) Pub. No.: US 2022/0031522 A1
(43) Pub. Date: Feb. 3, 2022

(54) Pull-Tab Bandage and Packaging

(71) Applicant: Best Bandage, LLC, Spokane, WA (US)

(72) Inventor: Brett E. Bajema, Colville, WA (US)

(73) Assignee: Best Bandage, LLC, Spokane, WA (US)

(21) Appl. No.: 17/380,809

(22) Filed: Jul. 20, 2021

Related U.S. Application Data
(60) Provisional application No. 63/059,110, filed on Jul. 30, 2020.

(51) Int. Cl. A61F 13/02; A61F 15/00

(52) U.S. Cl. CPC A61F 13/0259; A61F 15/001

(57) ABSTRACT

In aspects of a pull-tab bandage and packaging, the pull-tab bandage has an adhesive strip that includes a sterile pad, and a non-stick backing adheres to the adhesive strip and covers the sterile pad. The non-stick backing is removable from the adhesive strip for application of the sterile pad and the adhesive strip. The pull-tab bandage also has a pull-tab as an extension of the adhesive strip that extends beyond the packaging that encloses and seals around the pull-tab bandage. The pull-tab is perforated along a terminal break to separate from the adhesive strip. Upper and lower layers of the bandage packaging can be adhered together with a packaging adhesive, forming a sterile cavity around the pull-tab bandage. The pull-tab forms packaging gaps where the pull-tab extends beyond the packaging, and the packaging gaps can be sealed by the packaging adhesive to form the sterile cavity around the pull-tab bandage.

### RELATED APPLICATION

This application claims priority to U.S. Provisional Patent Application Ser. No. 63/059,110 filed Jul. 30, 2020 entitled “Pull-Tab Bandage and Packaging”, the disclosure of which is incorporated by reference herein in its entirety.

### BACKGROUND

Generally, most bandages can be difficult to open and apply, particularly while attempting to maintain sterility by not touching the sterile pad or the adhesive strip of a bandage. Traditional bandages require several steps to open and remove the packaging, remove the two non-stick backing pieces, and then apply the bandage. Given that bandages are primarily used to cover an injury and/or stop blood flow, the simple act of opening a bandage packaging and applying the bandage can take much longer than is preferred. Additionally, this multiple-step process to open a bandage packaging and apply the bandage can result in several instances of contaminating the sterile pad and the adhesive strip in a non-sterile environment and inadvertent contact. In a medical setting in particular, this poses a contamination risk for a patient when a healthcare practitioner is challenged to apply a bandage without touching the adhesive portion of the bandage. Notably, touching the adhesive portion of the bandage can lead to a higher risk of infection or cross contamination across patients when a contaminated portion of the bandage is applied to a patient's skin. Additionally, traditional bandage packaging generally results in several individual pieces of the packaging and non-stick backing that a person has to then collect and dispose of after applying a bandage.

### BRIEF DESCRIPTION OF THE DRAWINGS

Implementations of the techniques for a pull-tab bandage and packaging are described with reference to the following Figures. The same numbers may be used throughout to reference like features and components shown in the Figures:

FIG. 1 illustrates an example of a pull-tab bandage and packaging in accordance with one or more implementations as described herein.

FIG. 2 illustrates examples of alternative and/or additional features of a pull-tab bandage and packaging in accordance with one or more implementations as described herein.

FIG. 3 illustrates examples of alternative and/or additional features of a pull-tab bandage and packaging in accordance with one or more implementations as described herein.

FIGS. 4 and 5 illustrate example methods of a pull-tab bandage and the bandage packaging in accordance with one or more implementations of the techniques described herein.

### DETAILED DESCRIPTION

Implementations of a pull-tab bandage and packaging are described, and provides for a quick, easy-opening bandage packaging, as well as a simplified bandage application. Unlike traditional bandage packaging that requires several steps to open and remove the packaging, remove the non-stick backing pieces, and apply the bandage, the pull-tab bandage and packing described herein can be quickly opened to access the bandage for application, generally in half the time, while also producing fewer waste items. Additionally, the pull-tab bandage can be easily separated from all of the packaging and applied without contaminating or inadvertently contacting the sterile pad or the adhesive strip of the bandage. Notably, the packaging design described herein may also be implemented in similar concept for easy-opening applications other than bandages, including but not limited to adhesives, surgical instruments, medical requisites, veterinary supplies, catering supplies, horticultural consumables, cosmetics, and any other type of items that are generally packaged for use in a clean and sterile environment.

In aspects of a pull-tab bandage and packaging, an adhesive strip that includes a sterile pad, and a non-stick backing adheres to the adhesive strip and covers the sterile pad. The non-stick backing is removable from the adhesive strip for application of the sterile pad and the adhesive strip. In implementations, the non-stick backing is a one-piece backing that extends a full length and width of the adhesive strip inside of a sterile cavity formed by the packaging around the pull-tab bandage. Alternatively, the non-stick backing may be a two-piece backing with a first piece of the non-stick backing overlapping a second piece of the non-stick backing. The pull-tab bandage includes a pull-tab as an extension of the adhesive strip. The pull-tab is perforated along a terminal break to separate from the adhesive strip, or stated otherwise, the adhesive strip is perforated and the perforation demarks the pull-tab as the extension of the adhesive strip. As a terminal break, the perforation can be formed as an arc extending towards the pull-tab, and the terminal break allows for separation of the pull-tab from the adhesive strip after the application of the sterile pad and the adhesive strip.

Notably the pull-tab extends beyond the packaging that encloses and seals around the pull-tab bandage. The pull-tab forms packaging gaps where the pull-tab extends beyond the packaging, and the packaging gaps can be sealed with a packaging adhesive to form the sterile cavity around the pull-tab bandage. In implementations, the packaging gaps can be sealed by the packaging adhesive applied in a quantity sufficient to seal the packaging gaps and form the sterile cavity around the pull-tab bandage. Alternatively or in addition, the packaging gaps can be sealed by the packaging adhesive heat-pressed to seal the packaging gaps and form the sterile cavity around the pull-tab bandage. Further, an attached portion of the non-stick backing is attached to an inside of the packaging that is formed around the pull-tab bandage, and the attached portion is designed to peel the non-stick backing from the adhesive strip as the pull-tab is pulled to open the packaging.

In aspects of the bandage packaging for a pull-tab bandage, the bandage packaging includes a non-stick backing that adheres to an adhesive strip and covers a sterile pad of the pull-tab bandage. The non-stick backing is removable from the adhesive strip for application of the sterile pad and the adhesive strip. In implementations, the non-stick backing is a one-piece backing that extends a full length and width of the adhesive strip inside of the sterile cavity formed by the bandage packaging around the pull-tab bandage. Alternatively, the non-stick backing may be a two-piece backing with a first piece of the non-stick backing overlapping a second piece of the non-stick backing.

An upper layer of the bandage packaging can be adhered to a lower layer of the bandage packaging, forming the sterile cavity around the pull-tab bandage. In implementations, one or more attached portions of the non-stick backing are attached to an inside of the bandage packaging (e.g., attached inside of the upper layer and/or the lower layer of the packaging around the pull-tab bandage). The one or more attached portions are designed to peel the non-stick backing from the adhesive strip as the pull-tab is pulled to open the bandage packaging. The upper and lower layers of the bandage packaging form packaging gaps where the pull-tab of the pull-tab bandage extends beyond the bandage packaging, and the packaging gaps can be sealed by a packaging adhesive to form the sterile cavity around the pull-tab bandage. In implementations, the packaging gaps can be sealed by the packaging adhesive applied in a quantity sufficient to seal the packaging gaps and form the sterile cavity around the pull-tab bandage. Alternatively or in addition, the packaging gaps can be sealed by the packaging adhesive heat-pressed to seal the packaging gaps and form the sterile cavity around the pull-tab bandage.

In aspects of manufacturing and packaging a pull-tab bandage, a sterile pad can be added to an adhesive strip, and a non-stick backing adhered to the adhesive strip, which also covers the sterile pad. Generally, the non-stick backing may be a one-piece backing that extends a full length and width of the adhesive strip inside of a sterile cavity formed by the packaging around the pull-tab bandage. Alternatively, the non-stick backing may be a two-piece backing with a first piece of the non-stick backing overlapping a second piece of the non-stick backing. A terminal break is formed to demark a pull-tab of the adhesive strip, the pull-tab being an extension of the adhesive strip, and the pull-tab extending beyond the packaging that encloses and seals around the pull-tab bandage. In implementations, the terminal break can be a perforation that separates the pull-tab from the adhesive strip, and the terminal break allows for separation of the pull-tab from the adhesive strip after application of the sterile pad and the adhesive strip. In implementations, the perforation may be in the form of an arc extending towards the pull-tab.

Additionally, one or more attached portions of the non-stick backing are attached or adhered to an inside of the bandage packaging that is formed around the pull-tab bandage. The one or more attached portions are designed to peel the non-stick backing from the adhesive strip as the pull-tab is pulled to open the packaging. Generally, an upper layer of the packaging can be adhered to a lower layer of the packaging, such as with a packaging adhesive to form the sterile cavity around the pull-tab bandage. The packaging gaps that are formed by the pull-tab extending beyond the packaging are sealed. In implementations, the packaging gaps can be sealed by the packaging adhesive to form the sterile cavity around the pull-tab bandage, such as by the packaging adhesive applied in a quantity sufficient to seal the packaging gaps. Alternatively or in addition, the packaging gaps can be sealed by the packaging adhesive heat-pressed to seal the packaging gaps and form the sterile cavity around the pull-tab bandage.

While features and concepts of a pull-tab bandage and packaging can be implemented in any number of different environments and/or configurations, implementations of a pull-tab bandage and packaging are described in the context of the following example descriptions and methods.

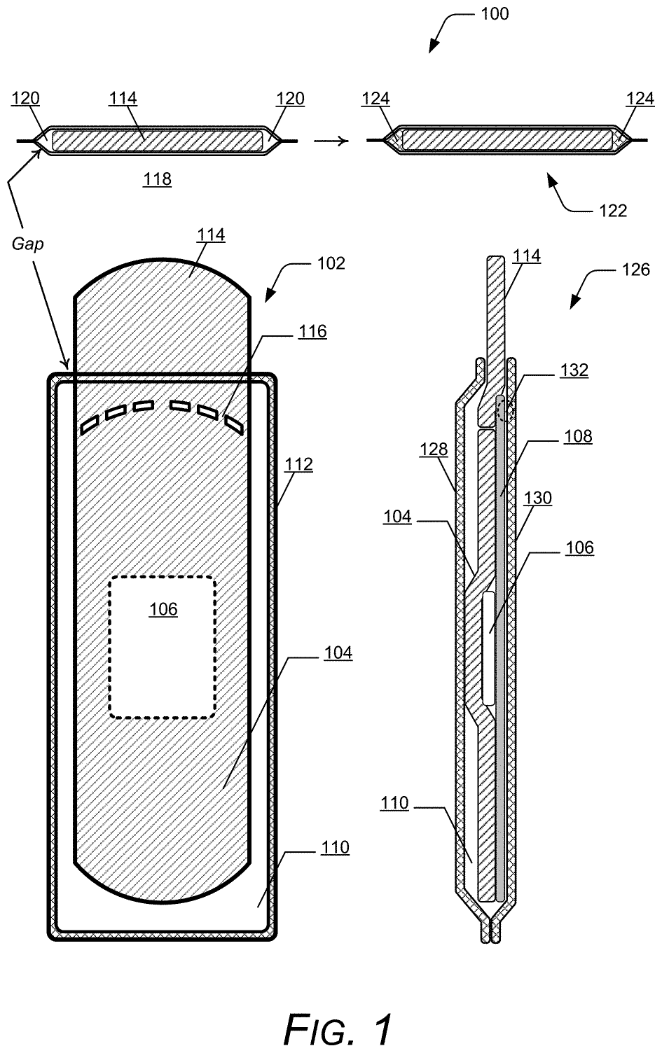

FIG. 1 illustrates an example 100 of a pull-tab bandage 102 and packaging, as described herein. In this example 100, the pull-tab bandage 102 may be designed as any type of an adhesive-backed bandage, usable in a multitude of environments for injury mitigation and/or as a skin or injury covering. The pull-tab bandage 102 has an adhesive strip 104 that includes a sterile pad 106, and a non-stick backing 108 adheres to the adhesive strip 104 and covers the sterile pad 106. The non-stick backing 108 is removable from the adhesive strip 104 for application of the sterile pad 106 and the adhesive strip. In implementations, the non-stick backing 108 is a one-piece backing that extends a full length and width of the adhesive strip 104 inside of a sterile cavity 110 formed by the packaging 112 around the pull-tab bandage. Alternatively, the non-stick backing 108 may be a two-piece backing with a first piece of the non-stick backing overlapping a second piece of the non-stick backing to cover the sterile pad 106.

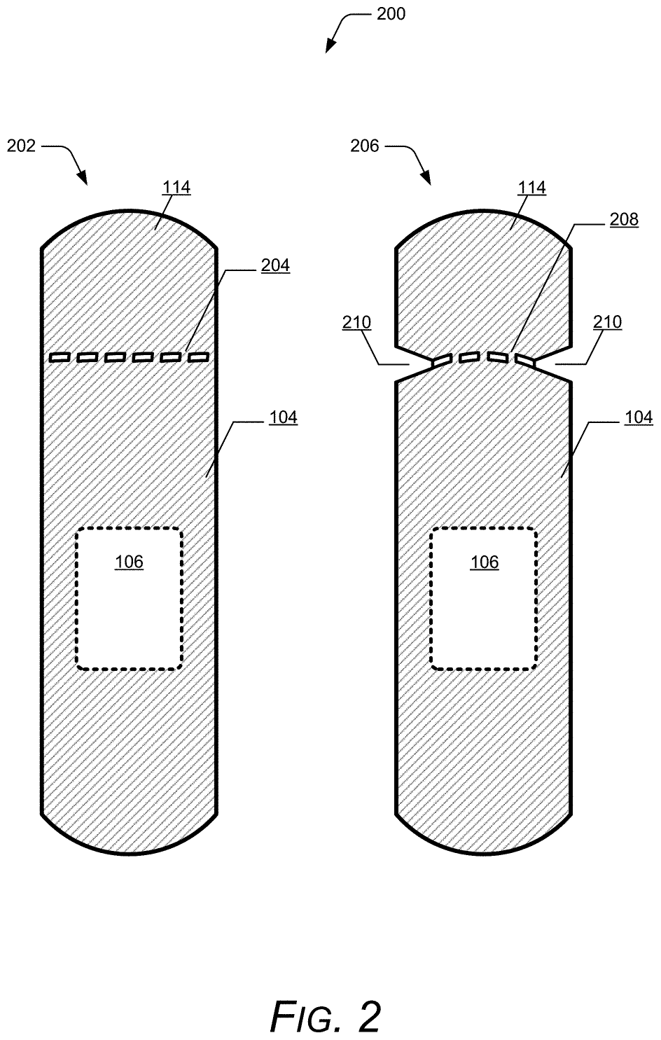

The pull-tab bandage 102 includes a pull-tab 114 as an extension of the adhesive strip 104. The pull-tab 114 can be perforated along a terminal break 116 to separate from the adhesive strip 104, or stated otherwise, the adhesive strip 104 is perforated and the perforation demarks the pull-tab 114 as the extension of the adhesive strip. As a terminal break 116, the perforation can be formed as an arc extending towards the pull-tab (as shown in this example 100), and the terminal break 116 allows for separation of the pull-tab 114 from the adhesive strip 104 after the application of the sterile pad and the adhesive strip. FIG. 2 further illustrates examples 200 of alternative and/or additional features of a pull-tab bandage and packaging, as described herein, and specifically, alternate designs of the terminal break used to separate the pull-tab 114 from the adhesive strip 104 after the application of the sterile pad and the adhesive strip. As shown in the example 202, a terminal break 204 may be an approximate perpendicular perforation across the pull-tab bandage 102 to separate the pull-tab 114 and the adhesive strip 104. Alternatively, as shown in the example 206, a terminal break 208 may be implemented as a perforation with notched edges 210, which may facilitate an easier separation of the pull-tab 114 from the adhesive strip 104.

Returning to the discussion of FIG. 1 and the example 100, this design of the pull-tab 114 as an extension of the adhesive strip 104 mitigates a design and/or manufacturing challenge, and facilitates ease of manufacturing by having the pull-tab 114 made of the same material as the adhesive strip 104. The non-adhesive pull-tab 114 is made of the same material as the bandage adhesive strip 104 and extends beyond the packaging 112, exposing a portion of the pull-tab bandage 102 which can be grasped to aid in opening the external packaging.

Notably the pull-tab 114 extends beyond the packaging 112 that encloses and seals around the pull-tab bandage. As shown in a top view 118, the pull-tab 114 forms packaging gaps 120 where the pull-tab extends beyond the packaging 112, and as further shown at 122, the packaging gaps 120 can be sealed with a packaging adhesive 124 to form the sterile cavity 110 around the pull-tab bandage 102. In implementations, the packaging gaps 120 can be sealed by the packaging adhesive 124 applied in a quantity sufficient to seal the packaging gaps and form the sterile cavity 110 around the pull-tab bandage. Alternatively or in addition, the packaging gaps 120 can be sealed by the packaging adhesive 124 being heat-pressed to seal the packaging gaps and form the sterile cavity 110 around the pull-tab bandage. Although the pull-tab 114 is shown and described herein as extending beyond the packaging 112, it should be noted that the entirety of the pull-tab bandage 102, to include the pull-tab 114, can be fully enclosed in the packaging 112 in an alternate configuration of the overall design and packaging of the pull-tab bandage.

As shown in the side view 126 of the pull-tab bandage 102 and packaging, an upper layer 128 of the bandage packaging 112 can be adhered to a lower layer 130 of the bandage packaging, forming the sterile cavity 110 around the pull-tab bandage. In implementations, an attached portion of the non-stick backing 108 is attached to an inside of the bandage packaging as shown at 132 (e.g., attached inside of the lower layer 130 of the packaging around the pull-tab bandage). The attached portion at 132 is designed to peel the non-stick backing 108 from the adhesive strip 104 as the pull-tab 114 is pulled to open the bandage packaging 112. In alternate implementations, the non-stick backing 108 may be attached to the inside of the bandage packaging (e.g., attached inside of the upper layer and/or the lower layer of the packaging around the pull-tab bandage) at more than one location. As noted above, the upper layer 128 and the lower layer 130 of the bandage packaging form the packaging gaps 120 where the pull-tab 114 of the pull-tab bandage 102 extends beyond the bandage packaging 112, and the packaging gaps 120 can be sealed by the packaging adhesive 124 to form the sterile cavity 110 around the pull-tab bandage.

In aspects of manufacturing and packaging the pull-tab bandage 102, the sterile pad 106 can be added to the adhesive strip 104, and the non-stick backing 108 adhered to the adhesive strip, which also covers the sterile pad. Generally, the non-stick backing 108 may be a one-piece backing that extends a full length and width of the adhesive strip 104 inside of the sterile cavity 110 formed by the packaging 112 around the pull-tab bandage. Alternatively, the non-stick backing 108 may be a two-piece backing with a first piece of the non-stick backing overlapping a second piece of the non-stick backing, and covering the sterile pad 106. The terminal break 116 is formed to demark the pull-tab 114 of the adhesive strip 104, with the pull-tab being an extension of the adhesive strip, and the pull-tab extending beyond the packaging 112 that encloses and seals around the pull-tab bandage. In implementations, the terminal break 116 can be a perforation that separates the pull-tab 114 from the adhesive strip 104, and the terminal break 116 allows for separation of the pull-tab from the adhesive strip after application of the sterile pad and the adhesive strip. In implementations, the perforation may be in the form of an arc extending towards the pull-tab, as shown in this example.

Additionally, one or more attached portions of the non-stick backing 108 are attached or adhered to an inside of the bandage packaging (e.g., the lower layer 130 of the packaging) that is formed around the pull-tab bandage, such as shown at 132. The one or more attached portions are designed to peel the non-stick backing 108 from the adhesive strip 104 as the pull-tab 114 is pulled to open the packaging. Generally, the upper layer 128 of the packaging can be adhered to the lower layer 130 of the packaging, such as with the packaging adhesive to form the sterile cavity 110 around the pull-tab bandage. The packaging gaps 120 that are formed by the pull-tab 114 extending beyond the packaging are sealed. In implementations, the packaging gaps 120 can be sealed by the packaging adhesive 124 to form the sterile cavity 110 around the pull-tab bandage, such as by the packaging adhesive 124 applied in a quantity sufficient to seal the packaging gaps. Alternatively or in addition, the packaging gaps 120 can be sealed by the packaging adhesive 124 being heat-pressed to seal the packaging gaps and form the sterile cavity around the pull-tab bandage.

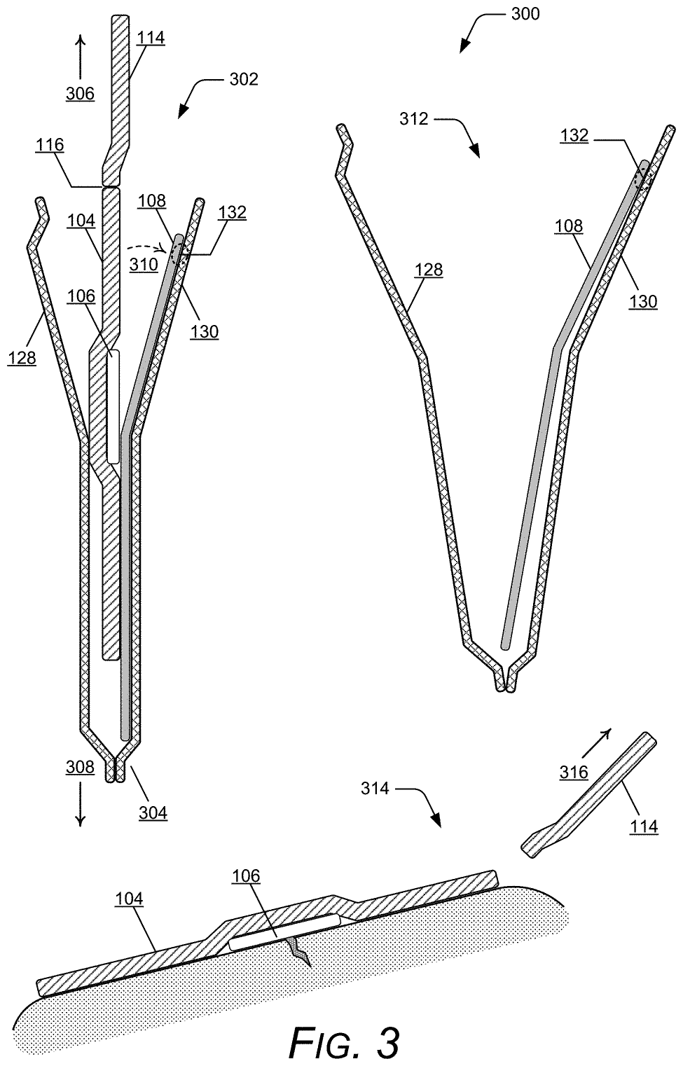

FIG. 3 illustrates examples 300 of alternative and/or additional features of a pull-tab bandage and packaging, as described herein, such as using the pull-tab bandage 102 as shown and described with reference to FIGS. 1 and 2. As shown in an example 302, a user of the pull-tab bandage 102 may grasp and pull on the pull-tab 114 of the pull-tab bandage that extends beyond the bandage packaging 112, and also grasp and hold onto the bandage packaging 112 opposite the pull-tab, such as at or near position 304 (e.g., at the bottom of the pull-tab bandage 102 relative to the illustration shown in FIG. 3). The sealed packaging 112 then separates responsive to the opposing forces, such as the first force 306 resulting from the grasp and pull on the pull-tab 114 that extends beyond the bandage packaging, and the second force 308 resulting from the grasp and hold on the bandage packaging opposite the pull-tab (e.g., at or near the position 304).

As the sealed bandage packaging 112 separates, the non-stick backing 108 is peeled from the adhesive strip 104, and the pull-tab bandage 102 is separated from the bandage packaging as shown at 310. For example, attached portion of the non-stick backing 108 that is attached to the inside of the bandage packaging at 132 (e.g., attached to the lower layer 130 of the packaging) is designed to peel the non-stick backing 108 from the adhesive strip 104 as the pull-tab 114 is pulled to open the packaging. As shown at 312, the bandage packaging and non-stick backing 108 are removed from the pull-tab bandage 102 as one piece, and can be easily discarded.

As further shown at 314, the sterile pad 106 can be adhered in place with the adhesive strip 104, such as over an injury to a user's skin, and the adhesive strip 104 holds the sterile pad 106 in place. The terminal break 116 then allows the pull-tab 114 to be separated from the adhesive strip 104 along the terminal break responsive to a grasp and pull 316 on the pull-tab 114 of the pull-tab bandage. After the placement of the pull-tab bandage 102, the non-adhesive pull-tab portion is easily removed from the adhesive strip 104 of the bandage, such as when torn, broken, twisted, ripped, etc. off of the main portion of the adhesive bandage. The two remnants, being the pull-tab 114 and the bandage packaging shown at 312, can then be discarded. Notably, because of the design, both pieces of the external packaging and the non-stick backing 108 can be continuously held in one hand while the pull-tab bandage 102 is applied with the other hand. Accordingly, the bandage can be opened, applied, and the packaging plus pull-tab 114 can be disposed of without ever having to release the original grip from opening the bandage packaging.

In aspects of the described pull-tab bandage and packaging, the sequence of the packaging 112 being opened includes the packaging adhesive 124 that seals the packaging gaps 120 first releasing and allowing the packaging to begin opening. In conjunction with the pull-tab bandage 102 being removed from the packaging as the pull-tab 114 is pulled, the non-stick backing 108 that is attached to the inside of the lower layer 130 of the packaging begins to peel away from the adhesive strip 104. Then, after the adhesive strip 104 is adhered to a surface (e.g., a user's skin) to hold the sterile pad 106 in position, the terminal break 116 allows the pull-tab 114 to be separated from the adhesive strip 104 along the perforation. Accordingly, the terminal break 116 is designed to have a stronger tensile strength (or bond strength) relative to the tensile strength (or bond strength) of the packaging adhesive 124 that seals the packaging gaps 120 to facilitate a user being able to pull on the pull-tab 114 and remove the pull-tab bandage 102 from the packaging 112 without the pull-tab 114 tearing away from the adhesive strip 104. Notably, the pull-tab 114 stays attached to the adhesive strip 104 throughout the process of first opening the bandage packaging, and second applying the bandage on a surface, such as adhering the adhesive strip 104 of the bandage on a user's skin.

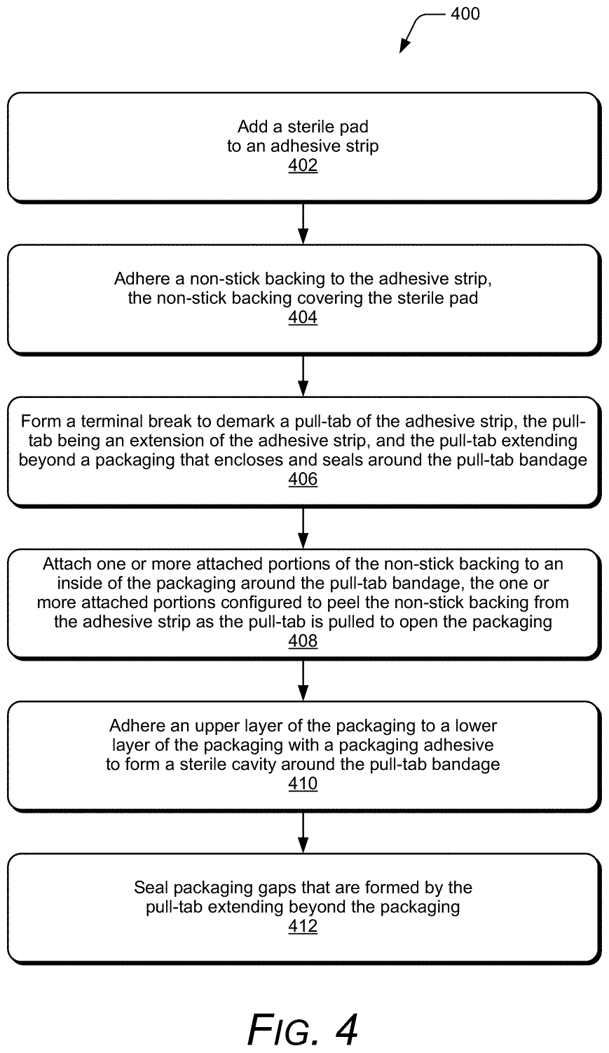

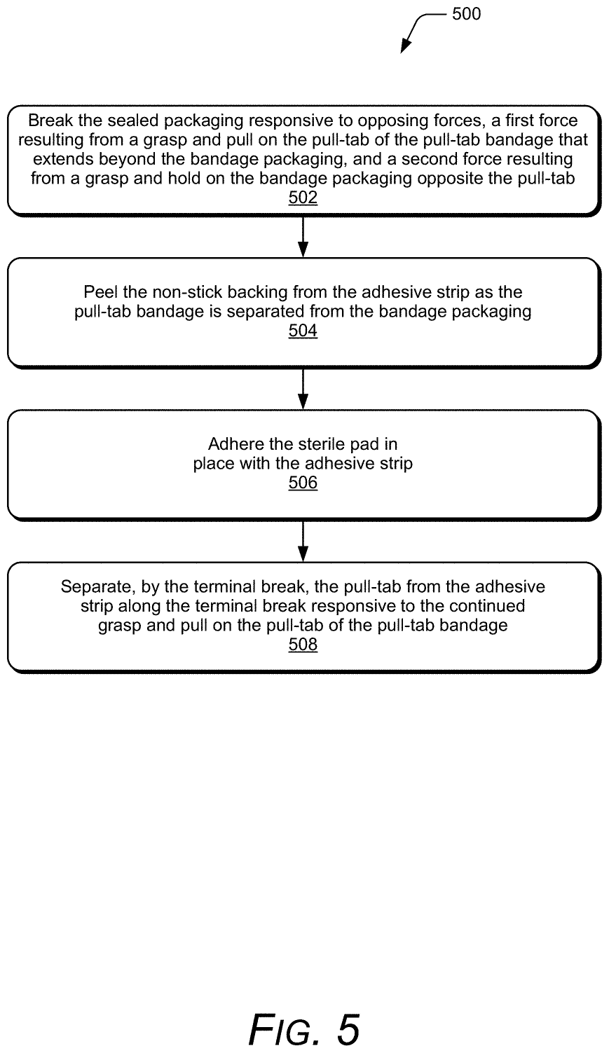

Example methods 400 and 500 are described with reference to respective FIGS. 4 and 5 in accordance with implementations of a pull-tab bandage and packaging.

FIG. 4 illustrates example method(s) 400 of a pull-tab bandage and packaging. The order in which the method is described is not intended to be construed as a limitation, and any number or combination of the described method operations can be performed in any order to perform a method, or an alternate method.

At 402, a sterile pad is added to an adhesive strip. For example, the sterile pad 106 is added, attached, or otherwise adhered to the adhesive strip 104. At 404, a non-stick backing is adhered to the adhesive strip, the non-stick backing covering the sterile pad. For example, the non-stick backing 108 is adhered to the adhesive strip 104 and covers the sterile pad 106. In implementations, the non-stick backing 108 is a one-piece backing that extends a full length and width of the adhesive strip 104 inside of a sterile cavity 110 formed by the packaging 112 around the pull-tab bandage. Alternatively, the non-stick backing 108 may be a two-piece backing with a first piece of the non-stick backing overlapping a second piece of the non-stick backing to cover the sterile pad 106.

At 406, a terminal break is formed to demark a pull-tab of the adhesive strip, the pull-tab being an extension of the adhesive strip, and the pull-tab extending beyond a packaging that encloses and seals around the pull-tab bandage. For example, the terminal break 116 is formed to demark and create the pull-tab 114 of the adhesive strip 104, and the pull-tab 114 is an extension of the adhesive strip 104. Notably, the pull-tab 114 extends beyond the packaging 112 that encloses and seals around the pull-tab bandage 102. In implementations, the terminal break 116 is a perforation that allows separation of the pull-tab 114 from the adhesive strip 104 after application of the sterile pad and the adhesive strip. Further, the perforation of the terminal break 116 may be formed as an arc extending towards the pull-tab section of the adhesive strip.

At 408, one or more attached portions of the non-stick backing are attached to an inside of the packaging around the pull-tab bandage, the one or more attached portions configured to peel the non-stick backing from the adhesive strip as the pull-tab is pulled to open the packaging. For example, an attached portion of the non-stick backing 108 is attached to an inside of the bandage packaging as shown at 132 (e.g., attached inside of the lower layer 130 of the packaging around the pull-tab bandage). The attached portion at 132 is designed to peel the non-stick backing 108 from the adhesive strip 104 as the pull-tab 114 is pulled to open the bandage packaging 112. In alternate implementations, the non-stick backing 108 may be attached to the inside of the bandage packaging (e.g., attached inside of the upper layer and/or the lower layer of the packaging around the pull-tab bandage) at more than one location.

At 410, an upper layer of the packaging is adhered to a lower layer of the packaging with a packaging adhesive to form a sterile cavity around the pull-tab bandage. For example, the upper layer 128 of the bandage packaging 112 can be adhered to the lower layer 130 of the bandage packaging with the packaging adhesive, forming the sterile cavity 110 around the pull-tab bandage 102.

At 412, packaging gaps that are formed by the pull-tab extending beyond the packaging are sealed. For example, the upper layer 128 and the lower layer 130 of the bandage packaging form the packaging gaps 120 where the pull-tab 114 of the pull-tab bandage 102 extends beyond the bandage packaging 112, and the packaging gaps 120 can be sealed by the packaging adhesive 124 to form the sterile cavity 110 around the pull-tab bandage. In implementations, the packaging gaps 120 can be sealed by the packaging adhesive 124 applied in a quantity sufficient to seal the packaging gaps and form the sterile cavity 110 around the pull-tab bandage. Alternatively or in addition, the packaging gaps 120 can be sealed by the packaging adhesive 124 being heat-pressed to seal the packaging gaps and form the sterile cavity 110 around the pull-tab bandage.

FIG. 5 illustrates example method(s) 500 of a pull-tab bandage and packaging. The order in which the method is described is not intended to be construed as a limitation, and any number or combination of the described method operations can be performed in any order to perform a method, or an alternate method.

At 502, the sealed packaging breaks responsive to opposing forces, a first force resulting from a grasp and pull on the pull-tab of the pull-tab bandage that extends beyond the bandage packaging, and a second force resulting from a grasp and hold on the bandage packaging opposite the pull-tab. For example, a user of the pull-tab bandage 102 can grasp and pull on the pull-tab 114 of the pull-tab bandage that extends beyond the bandage packaging 112, and also grasp and hold onto the bandage packaging 112 opposite the pull-tab, such as at or near the bottom of the pull-tab bandage. The sealed packaging 112 then separates responsive to the opposing forces, such as the first force 306 resulting from the grasp and pull on the pull-tab 114 that extends beyond the bandage packaging, and the second force 308 resulting from the grasp and hold on the bandage packaging opposite the pull-tab, such as near the bottom of the pull-tab bandage.

At 504, the non-stick backing is peeled from the adhesive strip as the pull-tab bandage is separated from the bandage packaging. For example, the one or more attached portions of the non-stick backing 108 that is attached to the inside of the bandage packaging (e.g., attached to the lower layer 130 of the packaging) peels the non-stick backing 108 from the adhesive strip 104 as the pull-tab 114 is pulled to open the sealed bandage packaging 112. Notably, the bandage packaging and non-stick backing 108 are removed from the pull-tab bandage 102 as one piece, and can be easily discarded.

At 506, the sterile pad is adhered in place with the adhesive strip. For the sterile pad 106 can be adhered in place with the adhesive strip 104, such as over an injury to a user's skin, and the adhesive strip 104 holds the sterile pad 106 in place. At 508, the terminal break allows the pull-tab to separate from the adhesive strip along the terminal break responsive to the continued grasp and pull on the pull-tab of the pull-tab bandage. For example, the terminal break 116 then allows the pull-tab 114 to be separated from the adhesive strip 104 along the terminal break responsive to the grasp and pull 316 on the pull-tab 114 of the pull-tab bandage. After the placement of the pull-tab bandage 102, the non-adhesive pull-tab portion is easily removed from the adhesive strip 104 of the bandage, such as when torn, broken, twisted, ripped, etc. off of the main portion of the adhesive bandage.

Although implementations of a pull-tab bandage and packaging have been described in language specific to features and/or methods, the appended claims are not necessarily limited to the specific features or methods described. Rather, the specific features and methods are disclosed as example implementations of a pull-tab bandage and packaging, and other equivalent features and methods are intended to be within the scope of the appended claims. Further, various different examples are described and it is to be appreciated that each described example can be implemented independently or in connection with one or more other described examples. Additional aspects of the techniques, features, and/or methods discussed herein relate to one or more of the following claims.

1. A pull-tab bandage, comprising:
an adhesive strip that includes a sterile pad;
a non-stick backing that adheres to the adhesive strip and covers the sterile pad, the non-stick backing removable from the adhesive strip for application of the sterile pad and the adhesive strip; and
a pull-tab as an extension of the adhesive strip that extends beyond a packaging that encloses and seals around the pull-tab bandage, the pull-tab forming packaging gaps where the pull-tab extends beyond the packaging, and the packaging gaps being sealed with a packaging adhesive to form a sterile cavity around the pull-tab bandage.

2. The pull-tab bandage as recited in claim 1, wherein the pull-tab is perforated along a terminal break to separate from the adhesive strip.

3. The pull-tab bandage as recited in claim 1, wherein the adhesive strip is perforated to demark the pull-tab that extends beyond the packaging that encloses and seals around the pull-tab bandage.

4. The pull-tab bandage as recited in claim 1, further comprising:
a perforation to demark the pull-tab as the extension of the adhesive strip, the perforation allowing separation of the pull-tab from the adhesive strip after the application of the sterile pad and the adhesive strip.

5. The pull-tab bandage as recited in claim 4, wherein the perforation is a terminal break in the form of an arc extending towards the pull-tab.

6. The pull-tab bandage as recited in claim 1, wherein the packaging gaps are sealed by the packaging adhesive applied in a quantity sufficient to seal the packaging gaps and form the sterile cavity around the pull-tab bandage.

7. The pull-tab bandage as recited in claim 1, wherein the packaging gaps are sealed by the packaging adhesive heat-pressed to seal the packaging gaps and form the sterile cavity around the pull-tab bandage.

8. The pull-tab bandage as recited in claim 1, wherein the non-stick backing is a one-piece backing that extends a full length and width of the adhesive strip inside of the sterile cavity formed by the packaging around the pull-tab bandage.

9. The pull-tab bandage as recited in claim 1, wherein the non-stick backing is a two-piece backing with a first piece of the non-stick backing overlapping a second piece of the non-stick backing.

10. The pull-tab bandage as recited in claim 1, wherein an attached portion of the non-stick backing is attached to an inside of the packaging around the pull-tab bandage, the attached portion configured to peel the non-stick backing from the adhesive strip as the pull-tab is pulled to open the packaging.

11. A bandage packaging, comprising:
a non-stick backing that adheres to an adhesive strip and covers a sterile pad of a pull-tab bandage, the non-stick backing removable from the adhesive strip for application of the sterile pad and the adhesive strip; and
an upper layer of the bandage packaging adhered to a lower layer of the bandage packaging forming a sterile cavity around the pull-tab bandage, the upper and lower layers of the bandage packaging forming packaging gaps where a pull-tab of the pull-tab bandage extends beyond the bandage packaging, and the packaging gaps are sealed by a packaging adhesive to form the sterile cavity around the pull-tab bandage.

12. The bandage packaging as recited in claim 11, wherein the pull-tab that extends beyond the bandage packaging is perforated along a terminal break to separate from the adhesive strip.

13. The bandage packaging as recited in claim 11, wherein the adhesive strip is perforated to demark the pull-tab that extends beyond the bandage packaging that encloses and seals around the pull-tab bandage.

14. The bandage packaging as recited in claim 11, wherein a perforation demarks the pull-tab as an extension of the adhesive strip, the perforation allowing separation of the pull-tab from the adhesive strip after the application of the sterile pad and the adhesive strip.

15. The bandage packaging as recited in claim 14, wherein the perforation is a terminal break in the form of an arc extending towards the pull-tab.

16. The bandage packaging as recited in claim 11, wherein the packaging gaps are sealed by the packaging adhesive applied in a quantity sufficient to seal the packaging gaps and form the sterile cavity around the pull-tab bandage.

17. The bandage packaging as recited in claim 11, wherein the packaging gaps are sealed by the packaging adhesive heat-pressed to seal the packaging gaps and form the sterile cavity around the pull-tab bandage.

18. The bandage packaging as recited in claim 11, wherein the non-stick backing is a one-piece backing that extends a full length and width of the adhesive strip inside of the sterile cavity formed by the bandage packaging around the pull-tab bandage.

19. The bandage packaging as recited in claim 11, wherein one or more attached portions of the non-stick backing are attached to an inside of the bandage packaging around the pull-tab bandage, the one or more attached portions configured to peel the non-stick backing from the adhesive strip as the pull-tab is pulled to open the packaging.

20. A method for a pull-tab bandage, the method comprising:
adding a sterile pad to an adhesive strip;
adhering a non-stick backing to the adhesive strip, the non-stick backing covering the sterile pad;
forming a terminal break to demark a pull-tab of the adhesive strip, the pull-tab being an extension of the adhesive strip and extending beyond a packaging that encloses and seals around the pull-tab bandage;
adhering an upper layer of the packaging to a lower layer of the packaging with a packaging adhesive to form a sterile cavity around the pull-tab bandage; and
sealing packaging gaps that are formed by the pull-tab extending beyond the packaging, the packaging gaps being sealed by the packaging adhesive to form the sterile cavity around the pull-tab bandage.
